(19) United States
(12) Patent Application Publication
(10) Pub. No.: US 2026/0338653 A1
(43) Pub. Date: Oct. 29, 2026

(54) Sampling Apparatus and Method and Testing Kit and Method

(71) Applicant: Howard Chandler, Avoca Beach (AU)

(72) Inventor: Howard Chandler, Avoca Beach (AU)

(73) Assignee: Howard Chandler, Avoca Beach (AU)

(21) Appl. No.: 18/997,318

(22) PCT Filed: Jun. 30, 2023
(86) PCT No.: PCT/AU2023/050607

§ 371 Date: Jan. 21, 2025

(30) Foreign Application Priority Data
Jul. 22, 2022 (AU) 2022902060

(51) Int. Cl. G01N 1/40; G01N 1/38; G01N 33/72

(52) U.S. Cl. CPC G01N 1/4077; G01N 1/38; G01N 33/721; G01N 2001/4088

(57) ABSTRACT

A kit comprising a protein containing substance for dispersion in a liquid, a sample collection device for collecting a sample from the liquid, the sample containing matter derived from faecal material, and a test device configured to detect one or more analytes in the sample. Also disclosed is a sample collection apparatus and method for collecting a sample derived from faecal material and a method for testing a sample derived from faecal material.

### TECHNICAL FIELD

The present disclosure relates to a sampling method and apparatus. In particular, the disclosure relates to a sampling apparatus and method which may be used to obtain a sample derived from faecal material. Further disclosed is a kit and method for testing a sample derived from faecal material for relevant biomarkers.

### BACKGROUND

Early detection of colorectal cancer may greatly decrease patient mortality. Detection of blood in faeces (also referred to herein as faecal material or stool) has been well established as an effective means of screening for early indications of colorectal cancer and its precursor polyps, adenomas or other bowl abnormalities.

Faecal Occult Blood Tests (FOBTs) are widely used for the screening of blood that may be present in a stool. Two types of FOBTs include an Immunochemical FOBT (iFOBT), also known as a Faecal Immunochemical Test (FIT), and a guaiac FOBT. An iFOBT or FIT, involves the collection of a stool sample using a device such as a spoon or the like and storage of the stool in a container. The collection container is typically then returned to a lab or doctor for testing of the sample. Guaiac FOBTs require individuals to collect a stool sample in a container from each of two to three bowel movements. A device, such as an applicator stick, is then typically used to apply a smear to a card. Once dried, the card may be sent to a doctor or laboratory for testing. If a positive result of blood is detected in the stool sample, further testing, such as a colonoscopy, may be required to locate the source of bleeding and/or diagnose disease.

An Immunochemical FOBT (iFOBT), also known as a Faecal Immunochemical Test (FIT), is a type of FOBT that involves the collection of a sample of a stool, which is typically then returned to a lab or doctor for testing for the presence of blood, and in particular haemoglobin in the sample. In particular, quantified FITs (qFITs) are a type of FIT that is used to determine the amount of haemoglobin in the stool sample. More recent developments in FIT and iFOBT techniques, involve the collection of a sample of faecal material in toilet water containing stool. If a positive result of blood, and specifically haemoglobin is detected in the sample and/or the concentration of haemoglobin exceeds a threshold, further testing, such as a colonoscopy, may be required to locate the source of bleeding and/or diagnose disease.

Although these techniques are typically successful in detecting haemoglobin in stool, which may indicate colorectal cancer and/or its precursor polyps, a consistent impediment to effective screening has been the reluctance of most of the at-risk population to collect and submit an identified stool sample for analysis. For example, the Australian Government, through its National Bowel Cancer Screening program, provides free FITs to individuals aged between 50-74, yet only approximately 40% of the subjects participate. These tests are intended to be performed at home and require the subject to collect the sample of faecal matter themselves. It is observed that the participation rate of screening is very low across the eligible screening population. One impediment to a high participation rate, and therefore more effective screening, may be the reluctance of the eligible screening population to collect and submit a stool sample for analysis, due to the unpleasant nature of handling faecal matter to obtain a sample and the long wait time to receive a test result.

There may be further limitations associated with conventional sampling and testing techniques. Firstly, the sample collected may only contain minute quantities of blood that can become rapidly undetectable when diluted in water, possibly due to changes in or loss of the haemoglobin (Hb) analyte at high dilution. Secondly, only a very small portion (typically less than 1%) of an entire stool is sampled therefore yielding a sample that may not representative of the whole stool. This may lead to a false negative test result in instances where blood is present in the stool but not necessarily present in the small sample taken.

More recent developments have involved detecting blood from a stool in toilet water, in one case by using a brush to collect a sample of water from around the stool and apply it to a testing device. However, the use of such brush devices still requires a close encounter with the faecal matter and may not be effective in obtaining a sample representative of the whole stool.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure provides examples of methods, apparatuses and kits which may enable collection and/or testing of a sample derived from faecal matter (such as for colorectal cancer screening). In particular, one or more of the disclosed examples may be suitable for obtaining a sample, whilst reducing contact between a user collecting the sample and the faecal matter from which the sample is derived. One or more of the disclosed examples may enable determining of the presence of haemoglobin at a higher sensitivity than conventional FITs.

According to one aspect of the present disclosure, there is provided a method for collecting a sample derived from faecal material, the method comprising:
placing a liquid-permeable membrane across a liquid, which contains faecal material, to obscure the faecal material;
advancing a sample collection device towards the liquid-permeable membrane;
manipulating the sample collection device to stir or agitate the liquid, such as to disperse matter derived from faecal material in the liquid; and
collecting a sample, using the sample collection device, from the liquid that has passed through the liquid-permeable membrane,
wherein, the liquid-permeable membrane is of sufficient strength to substantially maintain its form during the step of stirring or agitation of the liquid.

In some embodiments, the liquid may comprise water. In some examples, the liquid may comprise toilet bowl water. In other examples, the liquid may be provided in an alternative container. In some embodiments, the matter derived from the faecal material may comprise occult blood.

In some embodiments, advancing the sample collection device towards the liquid-permeable membrane may comprise placing a portion of the sample collection device (for example a distal tip of the sample collection device) on the liquid-permeable membrane. The sample collection device may contact an upper surface (in use) of the liquid-permeable membrane. The sample collection device may be manipulated to stir or agitate the liquid while remaining on, or in contact with, the upper surface of the liquid-permeable membrane.

In some embodiments, the method may further comprise depressing a region of the liquid-permeable membrane into the liquid, using a distal tip of the sample collection device. Depressing the region of the liquid-permeable membrane may be performed prior to manipulating the sample collection device to stir or agitate the liquid. For example, the liquid-permeable membrane may be depressed by contacting and applying a pressure to the region of the liquid-permeable membrane. Depressing the region of the liquid-permeable membrane may partially or wholly submerge the region of the liquid-permeable membrane. When depressed, the liquid permeable-membrane (or a portion thereof) may fold and/or wrap around a distal portion of the sample collection device.

Agitation of the liquid may disperse matter derived from the faecal material into the liquid, such as the toiled bowl water. A portion of the liquid containing the dispersed matter derived from the faecal material may then pass through the liquid-permeable membrane prior to collection of the sample of the liquid using the collection device.

In some embodiments, the method may further comprise testing the sample. Testing the sample may comprise transferring the sample to an assay device, or test device. The terms “assay device” and “test device” are used interchangeably herein. In some embodiments, the assay device may comprise a test strip, such as a lateral flow test strip.

Testing the sample may comprise testing for the presence of one or more analytes in the sample. At least one of the analytes may be indicative of a pathological condition in the faecal material from which the sample is derived. In some embodiments, at least one analyte may be indicative of occult blood, for example, the at least one analyte may comprise haemoglobin. Additionally or alternatively, one or more analytes may be indicative of other pathological conditions. For example, one or more analytes may be indicative of one or more tumour derived antigens.

In some embodiments the sample collection device may comprise a pipette. The pipette may comprise a hollow flexible bulb and a hollow tube, wherein a proximal end of the hollow tube is in fluid communication with the hollow bulb, and a distal end of the tube defines an opening. The opening may be at or adjacent to the distal tip of the sample collection device. In other embodiments, the sample collection device may comprise a brush or a brush-like apparatus. The brush may comprise flexible or semi-flexible bristles.

The method described in any of the preceding paragraphs may be performed using a sample collection apparatus or kit according to embodiments of the present disclosure.

According to one aspect of the present disclosure, there is provided a sample collection apparatus for collecting a sample derived from faecal material, the sample collection apparatus comprising:
a liquid-permeable membrane configured for placement across a liquid containing faecal material to obscure the faecal material; and
a collection device configured to collect a sample from the liquid that has passed through the liquid-permeable membrane, the sample containing matter derived from faecal material,
wherein, the collection device is configured to be manipulated by a user to stir or agitate the liquid, such as to disperse matter derived from faecal material in the liquid, prior to collection of the sample, and
wherein, the liquid-permeable membrane is of sufficient strength to substantially maintain its form during stirring or agitation of the liquid.

In some embodiments, the liquid may comprise water. For example, the liquid may comprise toilet bowl water. The matter derived from faecal material may comprise occult blood.

In some embodiments, the collection device may comprise a pipette having any of the features previously disclosed. Similarly, in another embodiment, however, the collection device may be a brush or a brush-like apparatus having flexible or semi-flexible bristles. The collection device may be configured to collect a volume of liquid substantially corresponding to a volume required for testing the sample.

The liquid-permeable membrane may be configured to obscure the faecal material such that substantially all of the faecal material is hidden from the view of a user collecting the sample. For example, in some embodiments the liquid-permeable membrane may be sized and/or shaped to extend substantially across the entire surface area of the liquid within the toilet bowl. In some embodiments, the liquid-permeable membrane may be sized and/or shaped to cover the surface area of the liquid as well as contact the inner walls of the toilet bowl. In other embodiments, the liquid-permeable membrane may be sized to extend partially across the surface area of the liquid within the toilet bowl and/or to partially obscure the faecal material.

The liquid-permeable membrane may be configured to be substantially opaque such that the faecal material is not visible through the membrane.

The liquid-permeable membrane may be configured to fold or wrap around the collection device. For example, the liquid-permeable membrane may fold around the collection device when a region of the permeable membrane is depressed with the collection device. The liquid-permeable membrane may be sized and/or shaped such that, when the region of the permeable membrane is depressed with the collection device, the liquid-permeable membrane continues to obscure the faecal material.

In some embodiments the liquid-permeable membrane may comprise a positively charged material. Alternatively or additionally, the liquid-permeable membrane may comprise colour catcher or dye trapping agents. Examples of positively charged materials, colour catcher and dye trapping agents are described in International Application No. PCT/US2021/022910, the disclosure of which is hereby incorporated by reference in its entirety.

In some embodiments, the liquid-permeable membrane may be configured to be flushable.

In another aspect of the present disclosure, there is provided a kit for testing a sample derived from faecal material, the kit comprising;

a sample collecting apparatus according to embodiments described herein; and

an assay device adapted for detection of one or more analytes in the sample.

In some embodiments, the assay device may comprise a test strip, for example, a lateral flow test strip.

In another aspect, there is provided a method for collecting a sample derived from faecal material, the method comprising:
advancing a sample collection device, sheathed in a liquid-permeable membrane, towards a liquid, which contains faecal material;
manipulating the sample collection device to stir or agitate the liquid, such as to disperse matter derived from the faecal material in the liquid; and
collecting a sample, using the sample collection device, from the liquid that has passed through the liquid-permeable membrane,
wherein the liquid-permeable membrane is of sufficient strength to substantially maintain its form during the step of stirring or agitation of the liquid. The method may further comprise sheathing the sample collection device in the liquid permeable membrane. The sample collection device may be withdrawn from the liquid permeable membrane after collecting the sample.

In another aspect, there is provided a sample collection apparatus for collecting a sample derived from faecal material, the sample collection apparatus comprising:
a collection device; and
a liquid-permeable membrane configured for sheathing a distal end of the collection device,
wherein the collection device is configured to collect a sample from liquid that has passed through the liquid-permeable membrane, the sample containing matter derived from faecal material,
wherein, the collection device is configured to be manipulated by a user to stir or agitate the liquid, such as to disperse matter derived from faecal material in the liquid, prior to collection of the sample,
wherein, the liquid-permeable membrane is of sufficient strength to substantially maintain its form during stirring or agitation of the liquid.

According to one aspect of the present disclosure, there is provided a kit comprising:
a protein containing substance for dispersion in a liquid;
a sample collection device, for collecting a sample from the liquid, the sample containing matter derived from faecal material; and
a test device configured to detect one or more analytes in the sample.

In some embodiments, the liquid may comprise water. For example, the liquid may comprise toilet bowl water.

In some embodiments, the sample collection device may be configured to stir or agitate the liquid, for example to disperse the matter derived from the faecal material in the liquid. Additionally or alternatively, the sample collection device may be configured to stir or agitate the liquid, to disperse the protein containing substance in the liquid.

In one or more embodiments, the protein containing substance may be derived from milk. In one embodiment, the protein containing substance may comprise a powder derived from milk, in particular a dried milk powder. In one example the protein containing substance may be a homogenised dried milk powder. In another embodiment, the protein containing substance may be a substance in the form of a liquid, paste, tablet, mixture or combination thereof, derived from milk. In this embodiment, the protein containing substance may increase the opacity of the liquid, in particular causing the liquid to become substantially opaque. In yet another embodiment, the protein containing substance may be any other suitable substance containing a protein. The protein containing substance may increase the opacity of the liquid to which it is added. In yet another embodiment, the protein containing substance may be any other suitable substance, such as bovine serum albumin (BSA), which substantially maintains the transparency of the liquid and does not increase the opacity of the liquid. In another embodiment, the protein containing substance may be derived from a non-animal protein source, such as a plant-based protein powder.

In some embodiments of the kit, the sample collection device may be configured to collect a volume of the liquid substantially corresponding to a volume required to test the sample. In some embodiments, the sample collection device may comprise a pipette. The pipette may comprise a hollow flexible bulb and a hollow tube, wherein a proximal end of the hollow tube is in fluid communication with the hollow bulb, and a distal end of the tube defines an opening. The opening may be at or adjacent to the distal tip of the sample collection device. In other embodiments, the sample collection device may comprise a brush, or a brush-like apparatus. The brush may comprise flexible or semi-flexible bristles, in which a volume of liquid may be collected.

In some embodiments, at least one analyte of the one or more analytes may be indicative of a pathological condition. For example, at least one analyte may be representative of occult blood in the sample, such as haemoglobin. Additionally or alternatively, one or more analytes may be indicative of other pathological conditions. For example, one or more analytes may be indicative of one or more tumour derived antigens.

In some embodiments, the test device may comprise a faecal immunochemical test device. The test device may include a lateral flow test strip. Alternatively, the test device may comprise any other testing means suitable for testing the one or more analytes in the sample.

In some embodiments, the kit may comprise a liquid-permeable membrane. The sample collection device may be configured to collect a sample from liquid that has passed through the liquid-permeable membrane, the sample containing matter derived from faecal material. The liquid-permeable membrane may be of sufficient strength to substantially maintain its form during stirring or agitation of the liquid.

In some embodiments, the liquid-permeable membrane may be configured for placement across the liquid, for example across the surface of toilet bowl water, to at least partially obscure the faecal material. The liquid-permeable membrane may be configured to obscure the faecal material such that substantially all of the faecal material is hidden from the view of a user collecting the sample. For example, in some embodiments the liquid-permeable membrane may be sized and/or shaped to extend substantially across the entire surface area of the liquid within the toilet bowl. In some embodiments, the liquid-permeable membrane may be sized and/or shaped to cover the surface area of the liquid as well as contact the inner walls of the toilet bowl. In other embodiments, the liquid-permeable membrane may be sized to extend partially across the surface area of the liquid within the toilet bowl and/or to partially obscure the faecal material. The liquid-permeable membrane may be configured to be substantially opaque such that the faecal material is not visible through the membrane.

In some embodiments the liquid-permeable membrane may comprise a positively charged material. Alternatively or additionally, the liquid-permeable membrane may comprise colour catcher or dye trapping agents. Examples of positively charged materials, colour catcher and dye trapping agents are described in International Application No. PCT/US2021/022910, the disclosure of which is hereby incorporated by reference in its entirety.

According to one aspect of the present disclosure, there is provided a method for testing a sample derived from faecal material, the method including:
adding a protein containing substance to a liquid;
allowing the protein containing substance to disperse in the liquid;
adding faecal material to the liquid;
collecting a sample, using a sample collection device, from the liquid, the sample including matter derived from the faecal material; and
testing the sample, using a testing device, for one or more analytes in the sample.

The method may use a protein containing substance, sample collection device and testing device substantially as described in relation to one or more of the embodiments disclosed herein.

In some embodiments, the liquid may comprise water. For example, the liquid may comprise toilet bowl water. The matter derived from faecal material may comprise occult blood.

In some embodiments, the protein containing substance may be added to the liquid prior to adding the faecal material to the liquid. In other embodiments, the protein containing substance may be added to the liquid after adding the faecal material to the liquid. In some embodiments, the protein containing substance may be added prior to collecting the sample from the liquid. For example, the protein containing substance may be added to toilet bowl water prior to collecting a sample from the toilet bowl water. In such embodiments, the collected sample may include the matter derived from faecal material and a portion of the protein containing substance. In other embodiments, however, the protein containing substance may be added subsequent to collection of the sample. For example, a sample including the matter derived from the faecal material may be collected from the liquid and a protein containing substance may be then added to the liquid of the sample.

In some embodiments, the method may include stirring or agitating the liquid. Stirring or agitating the liquid may enhance dispersion of the matter derived from faecal material and/or the protein containing substance within the liquid. In some embodiments, the sample collection device may be used to stir or agitate the liquid. However, other suitable methods and/or implements for stirring or agitating the liquid may be used.

In some embodiments, the method may include actively dispersing the matter derived from faecal material in the liquid, prior to collecting the sample. In one embodiment, this may include stirring or agitating the liquid. For example, the method may include manipulating the sample collection device to stir or agitate the liquid, such as to disperse the matter derived from the faecal material in the liquid.

In some embodiments, allowing the protein containing substance to disperse in the liquid may include actively dispersing the protein containing substance in the liquid. In some embodiments, this may include stirring or agitating the liquid. For example, the method may include manipulating the sample collection device to stir or agitate the liquid, such as to disperse the protein containing substance in the liquid.

In some embodiments, the method may include actively dispersing the protein containing substance in the liquid, prior to collection of the sample. For example, the method may include actively dispersing the protein containing substance in the liquid prior to adding the faecal material and prior to dispersing the matter derived from faecal material. Alternatively, the method may include adding the faecal material to the liquid and dispersing the matter derived from the faecal material in the liquid prior to dispersing the protein containing substance in the liquid. Alternatively, the method may include simultaneously dispersing the matter derived from the faecal material and the protein containing substance in the liquid in the same action. In another example, the method may include actively dispersing the protein containing substance in the liquid of the sample, after collection of the sample. For example, the protein containing substance may be located within the sample collection device and arranged to come into contact with the sample during or subsequent to collection of the sample. For example, the protein containing substance may be provided within a pipette, such as in a hollow bulb of the pipette.

In some embodiments, the test device may comprise a faecal immunochemical test device. The test device may include a lateral flow test strip. Alternatively, the test device may comprise any other testing means suitable for testing the one or more analytes in the sample.

In some embodiments, the method may include providing a liquid-permeable membrane. The sample may be collected from liquid that has passed through the liquid-permeable membrane. In one example, providing the liquid-permeable membrane may include placing the liquid-permeable membrane across the liquid, to at least partially obscure the faecal material. In another example, providing the liquid-permeable membrane may include sheathing at least a distal end of the sample collection device. In some embodiments, the sample collection device may be sheathed with the permeable membrane prior to stirring or agitation of the liquid. The liquid-permeable membrane may be of sufficient strength to substantially maintain its form during stirring or agitation of the liquid. The method may include withdrawing the sample collection device from the liquid-permeable membrane prior to testing the sample.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments are now described with reference to the accompanying drawings, in which:

FIGS. 1A and 1B are flowcharts showing steps in a method for collecting a sample derived from faecal material according to embodiments of the present disclosure;

FIG. 2 is a front view of a sample collection device according to one embodiment of the present disclosure;

FIG. 3 shows a front view of a sample collection device sheathed in a liquid-permeable membrane according to one embodiment of the present disclosure;

FIG. 4 shows a sample collection apparatus according to one embodiment of the present disclosure, including the pipette of FIG. 2 and a liquid-permeable membrane;

FIG. 5 shows components of a kit for collecting and testing a sample derived from faecal material, according to one embodiment of the present disclosure, the kit comprising the sample collection apparatus of FIG. 4;

FIG. 6 shows a kit for collecting and testing a sample derived from faecal material according to one embodiment of the present disclosure, the kit including a protein based substance, a sample collection device and a test device;

FIG. 7A shows a front view of a packaging for the protein containing substance of FIG. 6 in a closed state according to one embodiment of the present disclosure;

FIG. 7B show a front view of the packaging of FIG. 7A an open state;

FIGS. 8A and 8B show a perspective view of a test device of the kit of FIG. 6, before and after testing a sample;

FIG. 9A shows a kit for collecting and testing a sample derived from faecal material according to one embodiment of the present disclosure, the kit including a protein based substance, a sample collection device, a test device and a liquid-permeable membrane;

FIG. 10A is a flowchart showing steps in a method for testing a sample derived from faecal material according to one embodiment of the present disclosure; and

FIG. 10B is a flowchart showing steps in a method for testing a sample derived from faecal material according to another embodiment of the present disclosure.

### DESCRIPTION OF EMBODIMENTS

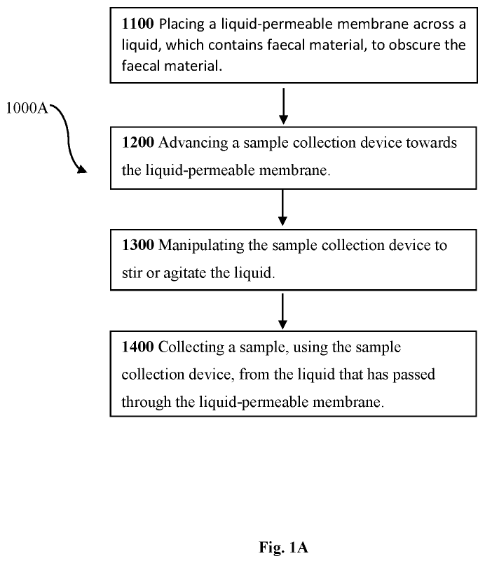

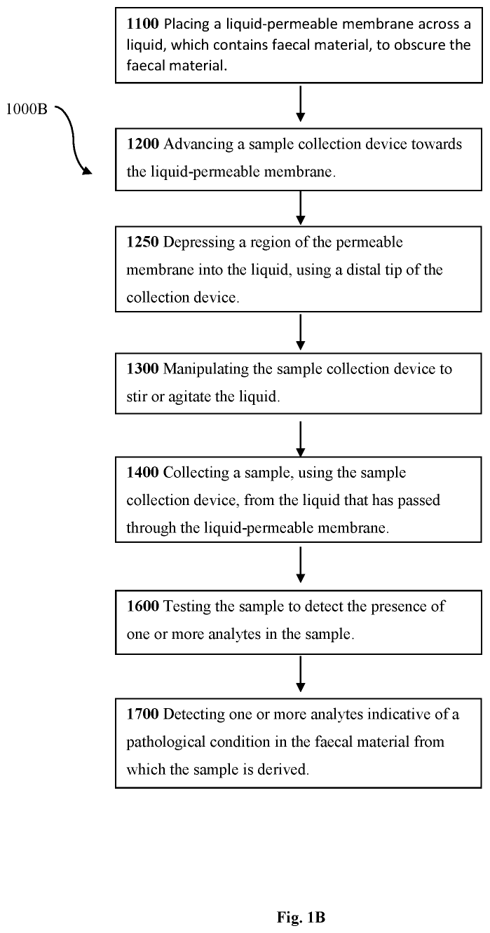

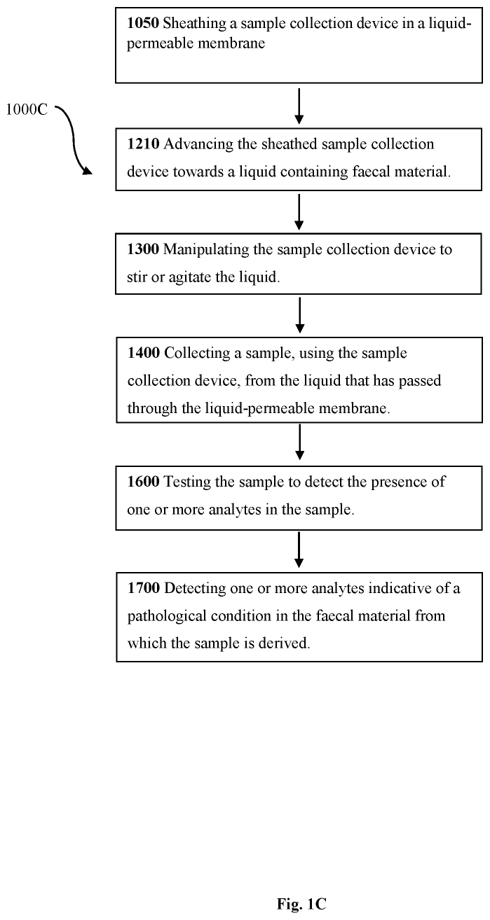

A method for collecting a sample derived from faecal material, according to the present disclosure is shown as flowchart 1000A in FIG. 1A. The method 1000A comprises placing a liquid-permeable membrane across a liquid, which contains faecal material, to obscure the faecal material 1100, advancing a sample collection device towards the liquid-permeable membrane 1200, manipulating the sample collection device to stir or agitate the liquid 1300, such as to disperse matter derived from faecal material in the liquid, and collecting a sample, using the sample collection device, from a portion of the liquid that has passed through the liquid-permeable membrane 1400.

Some embodiments comprise one or more further steps 1250, 1600 and 1700 of flowchart 1000B, illustrated in FIG. 1B. The relative positions of the method steps as depicted in flowcharts 1000A and 1000B should not be interpreted as limiting those steps to being performed in any particular order.

Examples of a sample collection device 200 and a liquid-permeable membrane 410, 420 suitable for use in the methods of FIGS. 1A to 1C are shown in FIGS. 2-5. The sample collection device 200 and the liquid-permeable membrane 410 and 420 as well as alternative embodiments, are disclosed in more detail below.

In one embodiment, the liquid across which the liquid permeable membrane is to be placed is water, in particular toilet bowl water. In this embodiment, placing the liquid-permeable membrane across a liquid thus comprises placing the liquid-permeable membrane in a toilet bowl which contains toilet bowl water and faecal material. The liquid-permeable membrane is thus placed to obscure the faecal material after defecation. In other embodiments, however, the liquid may be water in any other container suitable for receiving faecal material. Positioning the liquid permeable membrane in this manner creates a barrier between the user and the faecal material such that only liquid may pass through, preventing faecal material from being sampled. In addition, this will reduce the need for contact with or sight of the stool itself when retrieving the sample and thereby improve compliance of individuals conducting bowel cancer screening.

Stirring and agitation of the liquid promotes matter derived from the faecal material to mix or disperse in the liquid in which the faecal material is submerged. In one embodiment, the matter derived from faecal material is occult blood and, as such, contains haemoglobin. The stirring and agitation of matter derived from the faecal material in the surrounding liquid may enable the haemoglobin of the occult blood to disperse and may facilitate a more even distribution of haemoglobin in the liquid. This may increase the probability of collecting a sample that contains occult blood and haemoglobin, representative of the whole of the faecal material, even if the sample collected is small.

To this end, the method 1000B may further comprise step 1250 of depressing a region of the permeable membrane into the liquid, using a distal tip of the collection device. The pressure applied to contact and depress a region of the liquid-permeable membrane, may cause the liquid-permeable membrane to fold around a distal portion, adjacent the distal end D, of the collection device. Folding of the liquid-permeable membrane around the collection device may act as a further barrier between the collection device and the faecal material below the liquid-permeable membrane. This may optimise the chance that that the user avoids any direct encounter with the faecal material itself. Depressing the region of the permeable membrane may be performed prior to stirring or agitating the liquid. The folding of the liquid-permeable membrane around the collection device may also aid in the dispersion of the haemoglobin of the occult blood in the liquid during stirring and agitation, due to the increased mass provided to the distal end of the collection device.

The method shown in flowchart 1000B may further comprise a step 1600 of testing the sample to detect the presence of one or more analytes in the sample. In one embodiment, testing the sample involves transferring the collected sample of the liquid to a testing device, such as an assay device.

As further shown in flowchart 1000B, testing of the sample may further comprise detecting one or more analytes indicative of a pathological condition in the faecal material from which the sample is derived 1700. In one embodiment, screening involves testing for at least one analyte that is representative of occult blood, for example, testing for the presence of haemoglobin. Haemoglobin in the sample of liquid may be indicative of colorectal cancer or its precursor polyps. In another embodiment, however, the analyte or analytes may be representative of other pathological conditions, for example one or more tumour derived antigens. As mentioned above, stirring and agitating the liquid facilitates an even dispersion of the matter derived from faecal material within the liquid from which the sample is collected, such that the sample is representative of the whole stool. This is in contrast to direct sampling methods, which may collect a sample of faecal material that does not contain the analyte of interest, even though the analyte may be present within other portions of the stool. The presently disclosed methods and apparatus therefore increases the probability of detection of one or more analytes indicative of a pathological condition (for example, haemoglobin) if those analytes are present in the stool. This, ultimately, increases the sensitivity of the test.

The liquid permeable membrane 410, 420 in embodiments of the kit, sampling apparatus or method disclosed herein may include one or more of the features as described below.

Examples of a liquid permeable membrane 410 are shown in FIGS. 4, 5 and 9. The liquid-permeable membrane 410 may be configured to obscure the faecal material such that substantially all of the faecal material is hidden from the view of a user collecting the sample. While the liquid permeable membrane 410 in the figures has a rectangular shape, other shapes are also contemplated. In one embodiment, the liquid-permeable membrane 410 may be sized or shaped to cover the surface area of the liquid within the toilet bowl. In another embodiment, the liquid-permeable membrane 410 may be sized or shaped to cover the surface area of the liquid as well as contact the inner walls of the toilet bowl. In another embodiment, the liquid-permeable membrane 410 may be sized or shaped to extend partially across the surface area of the liquid within the toilet bowl and/or to partially obscure the faecal material. Partial or total obscuring of the faecal matter may minimise the requirement for close contact with the faecal matter and may serve to reduce user reluctance in performing the sampling.

The liquid-permeable membrane 410 may be configured to fold or wrap around the distal end of the sample collection device 200 when a region of the permeable membrane is depressed with the distal end D of the sample collection device 200. This may provide a means of filtering the liquid to allow the passage of liquid, while inhibiting the passage and collection of solid faecal material or other particulate contaminants during collection of the sample. This may reduce the risk of the user contacting faecal material and may optimise the performance of the test in the detection of the one or more analytes, such as haemoglobin. Alternatively, a liquid permeable membrane 420 may be provided in the form of a pouch or sheath, as described in further detail below.

The liquid-permeable membrane 410, 420 may be of sufficient strength to substantially maintain its form during stirring or agitation of the liquid. For example, the liquid-permeable membrane 410, 420 may have sufficient strength to withstand any rips, tears or other damage that may occur during stirring and/or agitation.

The liquid-permeable membrane 410, 420 may be configured to only allow the passage of liquid and matter derived from the faecal material which has been dissolved within the liquid, which, in the present embodiment, may be occult blood containing haemoglobin. As such, the liquid permeable membrane 410, 420 may prevent or substantially inhibit the passage of solid faecal material (or other particular contaminants) therethrough, thus inhibiting collection of any solid faecal material in the sample. This may optimise the performance of the test in the detection of the one or more analytes, such as haemoglobin.

In some embodiments, the liquid-permeable membrane 410, 420 is configured to be flushable. The use of the term “flushable” or variations such as “flushability” throughout the present disclosure, implies that a product or material, in particular the liquid-permeable membrane 410, 420, is configured to be disposable by flushing from a toilet bowl into the outflow and sewer or drainage pipes, without causing any blockage or damage to the sewage or drainage pipes through which it passes.

In some embodiments the liquid-permeable membrane 410, 420 may comprise a positively charged material. The liquid-permeable membrane may, additionally or alternatively, comprise a colour catcher or dye trapping agent. Colour catcher or dye trapping agents may remove any colour or capture any entities from the liquid, which contains faecal material, from passing through the liquid-permeable membrane that may negatively interfere with the test result. In the present disclosure, the term “colour catcher” or “dye trapping agent” implies a material, a material property or a product that has been treated in such a way so at to impede the transfer of any pigment that may seep into the liquid. In one embodiment, this implies that any pigments, colours, dyes or associated elements of the faecal material that may seep into in the liquid, which have passed through the liquid-permeable membrane, will be retained in the material of the liquid-permeable membrane as the liquid passes through.

The liquid-permeable membrane 410, 420 may be configured to be substantially opaque (for example, even when saturated by the liquid), such that the faecal material is not visible through the membrane. This may reduce the need for contact with and/or sight of the stool itself, which may improve user compliance in using the kit to conduct testing. In some embodiments, the liquid-permeable membrane 410, 420 may contain a colour pigment to further conceal the faecal material in the liquid. In one embodiment the collection device may comprise a pipette. In the embodiment illustrated in FIG. 2, the pipette 200 comprises a hollow flexible bulb 210 and a hollow tube 220. The hollow tube is fluidly connected to the bulb at its proximal end P and defines an opening 230 at its distal end D. In the illustrated embodiment, the opening 230 is at the distal end of the hollow tube, which is the distal tip of the collection device. The pipette may comprise graduations indicating the volume of liquid that can be held. In an alternative embodiment, the pipette may comprise a rotational portion and a plunger, wherein the rotational portion, when rotated, allows for the plunger volume to be adjusted to the desired volume for collection of the sample. The pipette and any of its components may be constructed from glass, plastic, rubber or combinations of such materials. Collection of a sample using a pipette may enable collection of a quantitative or semi quantitative sample. That is, use of pipette may enable collection of a controlled, consistent volume of liquid that is suitable for testing the sample.

In another embodiment, however, the collection device may be an alternative device suitable for collecting a sample of liquid, such as a brush or a brush-like apparatus having flexible or semi-flexible bristles at or adjacent a distal end. In this instance, the volume of liquid in the sample is collected in a semi-quantitative manner, in that the amount of liquid held within the interstitial spaces of the bristles of the sampling device will be a reasonably constant amount for any particular size and configuration of the sampling device.

In some embodiments, the sample collection device 200 may be configured to stir or agitate the liquid, for example by having sufficient strength to allow stirring or agitation of the liquid when held by the proximal end P. Stirring or agitating the liquid may disperse the matter derived from the faecal material in the liquid. Similarly, stirring or agitating the liquid or the liquid in the sample may enhance dispersing and/or dissolving the protein containing substance 100 in the liquid prior to collection of the sample, or in the liquid of the sample after collection of the sample.

In some embodiments, the assay device may be any assay device that is capable of detecting one or more analytes indicative of a pathological condition in the faecal material from which the sample is derived. The assay device may include a test strip such as a lateral flow test strip, also known as a immunochromatographic test strip or a rapid test, which allows the test to develop in a relatively short period of time, when the sample is introduced to the cartridge providing an accurate assay of the sample at the time of collection. The test results may then be read immediately, or at any time thereafter without risk of analyte loss through degradation during transport, as found in other tests. In another embodiment, testing of the sample may involve transporting the sample to a testing facility such as a laboratory for testing.

The method may be carried out using a sample collection apparatus. FIG. 4 shows a sample collection apparatus 520 according to the present disclosure, for collecting a sample derived from faecal material. In the illustrated embodiment, the sample collection apparatus 520 comprises a liquid-permeable membrane 410 and a collection device 200 embodying the features previously described, respectively. In this embodiment, the liquid-permeable membrane 410 is configured to obscure the faecal material such that substantially all of the faecal material is hidden from the view of a user collecting the sample, as previously described.

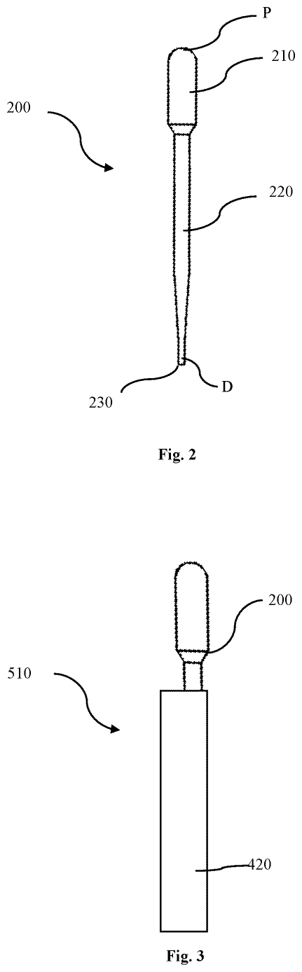

FIG. 3 shows another embodiment of a sample collection apparatus 510, in which the sample collection device 200 is sheathed in a liquid-permeable membrane 420. The liquid-permeable membrane 420 may comprise one or more features or characteristics as described herein in relation to liquid permeable membrane 410. In this embodiment, the liquid-permeable membrane 420 wraps around the hollow tube and distal tip of the pipette.

In this embodiment, the liquid-permeable membrane 420 may be in the form of a sleeve or a pouch into which the sample collection device 200 may be inserted. The liquid-permeable membrane 420 may be configured to enclose at least a distal end of the sample collection device 200. In the embodiment of FIG. 3, the liquid-permeable membrane 420 is configured to sheath the hollow tube 220 of the sample collection device from the distal end to a proximal region thereof in proximity to the bulb 210. In other embodiments, the liquid-permeable membrane 420 may enclose or sheath a smaller portion of the sample collection device 200, for example, at least a region adjacent to the distal tip D of the sample collection device 200.

The pipette 200 and membrane 420 may be used to carry out an alternative method of collecting a sample, as illustrated in method 1000C of FIG. 1C. The method may include a step 1050 of sheathing the sample collection device 200 in the liquid-permeable membrane 420. Alternatively, the sample collection device may be supplied pre-sheathed.

In this embodiment, the sheathed pipette 200 is advanced into the liquid (step 1210 in FIG. 1C) and used to stir or agitate the liquid and to collect a sample of liquid that has passed through the membrane 420, as previously described in relation to steps 1300 and 1400 of FIGS. 1A and 1B. The method may then include further steps 1600 and 1700 of testing the sample and detecting analytes, as previously described.

The liquid-permeable membrane 420 of the above described embodiment may not be large enough to substantially obscure the faecal material. However, the membrane 420 provides a means of filtering the liquid to inhibit the passage of faecal material and/or other contaminating particles through the membrane 420 during collection of the sample. The liquid-permeable membrane 420 may be configured to remain in the liquid once it is wet and the pipette 200 is withdrawn, for example by sliding off the distal end of the pipette 200.

FIG. 5 shows a kit 5000 for testing a sample derived from faecal material. In the illustrated embodiment, the kit 5000 comprises the sample collecting apparatus 520 comprising the liquid-permeable membrane 410 and sample collection device 200 as previously described. Alternatively, the kit 5000 may comprise the sample collection apparatus 510 as shown in FIG. 3. In alternative embodiments, the sample collection device may be any other device suitable for collecting a sample of liquid, such as a brush or a brush-like apparatus having flexible or semi-flexible bristles, as previously disclosed. The kit 5000 also comprises a test device such as assay device 5100 (or test device 300) adapted for detection of one or more analytes in the sample. The assay device may include any of the features and embody the principles previously described.

At least one analyte being detected in the assay device 5100 is indicative of a pathological condition in the faecal material from which the sample is derived. In one embodiment this analyte may be representative of occult blood, for example haemoglobin, in the sample. However, in another embodiment, this may be any other analyte indicative of a pathological condition, such as, for example, an analyte or analytes representing one or more tumour derived antigens.

Kits and methods according to embodiments of the present disclosure including a protein containing substance 100 are described in more detail below.

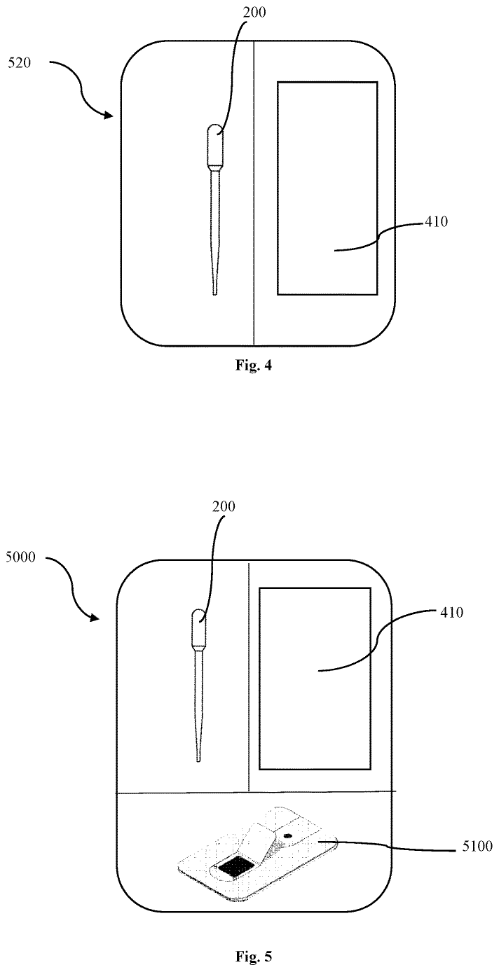

A kit 6000 according to one embodiment of the present disclosure is shown in FIG. 6. The kit 6000 comprises a protein containing substance 100 for dispersion in a liquid, a sample collection device 200, for collecting a sample from the liquid, the sample containing matter derived from faecal material and a test device 300, configured to detect one or more analytes in the sample. An alternative kit 7000, which may include a liquid permeable membrane 410 (or 420) is also described below. Methods for testing a sample derived from faecal material are described with reference to flowcharts 3000A, 3000B.

Examples of a protein containing substance 100 and a test device 300 suitable for inclusion in the kits 6000, 7000 and suitable for use in methods shown in flowcharts 3000A, 3000B disclosed herein, are shown in FIGS. 6 to 9A and are described in more detail below. The sample collection device 200 and liquid permeable membrane 410, 420 as used in the methods 3000A, 3000B or kits 6000, 7000 may include one or more of the features as described above with reference to FIGS. 2 to 5.

In one embodiment, the liquid from which the sample is to be collected is water. For example, the liquid may be toilet bowl water. The protein containing substance 100 may be dispersed in the water. For example, the protein containing substance 100 may be added to water in a toilet bowl either before or after addition of a stool to the water.

The protein containing substance 100 may be a substance capable of dispersion in water. In one embodiment, the protein containing substance 100 may be capable of dissolving in the toilet bowl water. Alternatively, the protein containing substance 100 may be capable of dispersing and/or dissolving in a liquid (such as water) in an alternative container, suitable for receiving faecal material.

In the embodiment shown in FIG. 6, the protein containing substance 100 is in the form of a powder. In another embodiment, the protein containing substance 100 may be provided in any other form capable of dispersing and/or dissolving in the liquid. For example, the protein containing substance 100 may include a liquid, paste, tablet, pill, mixture or combination thereof. In one embodiment, the protein containing substance 100 is a dried milk powder, such as a commercial whole milk powder or skim milk powder. In this embodiment, the dried milk powder may be formed using a number of powder processing techniques, including fat standardization, pasteurization, evaporation, homogenization and spray drying. The dried milk powder may be a homogenised dried milk powder.

In another embodiment, the protein containing substance 100 may be a liquid For example, the protein containing substance 100 may include a commercial milk product such as a whole milk, a reduced-fat milk or a combination thereof, including preservatives diluted in a liquid such as water. In some embodiments, the user may supply the protein containing substance 100 for addition to the liquid.

In another embodiment, the protein containing substance 100 may include a solid substance, such as a tablet or pill, derived from milk. In this embodiment, the tablet or pill may include a powder derived from milk, such as the dried milk powder as previously described and a dissolvable binder component, compressed into the form of a tablet or pill. Additionally or alternatively, the protein containing substance may include one or more dispersing agents. The one or more dispersing agents may comprise one or more active ingredients, such as sodium bicarbonate and/or citric acid, or other suitable dispersing agent. The dispersing agent may aid in dissolution and/or dispersion of the protein containing substance in the liquid, for example by providing an effervescent effect. When the tablet or pill is added to the liquid the dissolvable binder component and/or dispersing agent may function to break apart the components of the tablet upon sustained contact with the liquid, such that the protein containing substance 100 disperses and/or dissolves in the liquid. In another embodiment, the protein containing substance 100 may include one or more of a powder, paste, liquid, tablet, or mixture.

The protein containing substance 100 may be a substantially opaque substance. Addition of the protein containing substance 100 to a liquid may increase the opacity of the liquid in which it disperses and/or dissolves. In some embodiments, the protein containing substance 100 causes the liquid to become substantially opaque. In embodiments where the protein containing substance 100 is added to and disperses in water, in particular toilet bowl water, the liquid may change from substantially transparent to substantially opaque. The increased opacity of the liquid may at least partially obscure the contents of the liquid, which may enable at least partial obscuring of faecal material in the toilet bowl water.

The protein containing substance 100 may additionally or alternatively alter the consistency and/or colour of the liquid, providing a means to substantially obscure from the view of the user faecal material that is added to the liquid after defecation. In some embodiments, the colour and opacity of the protein containing substance 100 may be white and/or cloudy, as is typical of milk or milk-like products. As such, when the protein containing substance 100 is added to the liquid and disperses in the liquid, the liquid increases in opacity from a substantially transparent liquid to a cloudy, milky white liquid, substantially obscuring any faecal material from the view of the user. The protein containing substance 100 may include colourants or dyes that that may alter the colour and opacity of the liquid to which the protein containing substance is added, such that the liquid transitions from substantially transparent to a substantially opaque and coloured liquid.

Partial or total obscuring of the faecal material, for example by increasing the opacity and/or changing the colour of the liquid, may serve to reduce the visibility of the stool to the user and may decrease user reluctance in performing the sampling.

In alternative embodiments, the protein containing substance 100 may be an alternative powder, paste, liquid, tablet, mixture or combination thereof, containing a suitable protein. In some embodiments, the protein containing substance 100 may include bovine serum albumin (BSA). In some embodiments, addition of the protein containing substance 100 to the liquid may not change the opacity of the liquid, which may remain substantially transparent. This may be desirable in circumstances where a clear sample is required. This may be the case, for example, when using some quantified FITs, such as “OC-Sensors”.

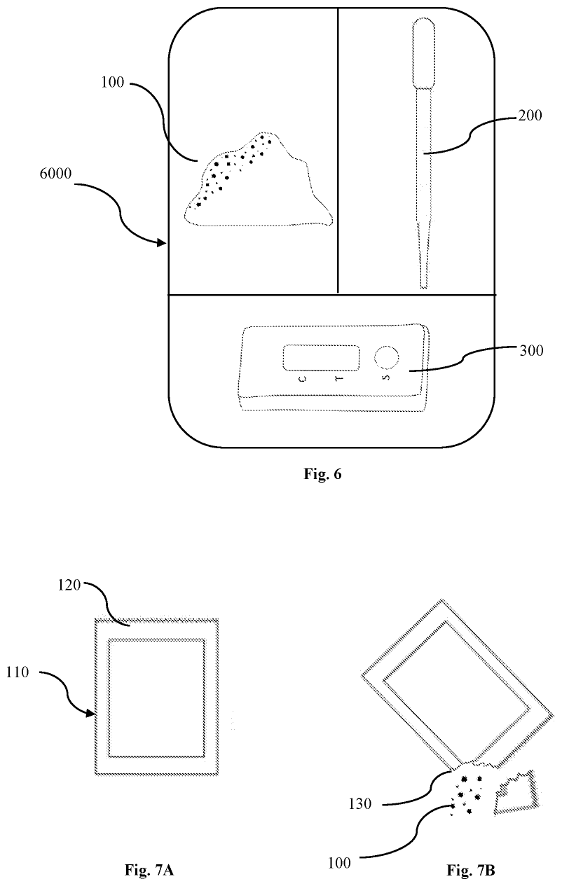

As shown in the embodiment depicted in FIG. 7A, the protein containing substance 100 may be packaged in a sachet 110, which may be provided in the kit 6000. In this embodiment, the sachet 110 comprises at least one seal 120 that is configured to appropriately seal the sachet, for example via an airtight seal at one end or around a periphery of the sachet, to inhibit exposure of the protein containing substance 100 to moisture and/or external air prior to use. Sealing the sachet may inhibit or reduce degradation or contamination of the protein containing substance 100 before use.

As shown in FIG. 7B, the sachet 110 may be torn, for example in proximity to the seal (for example, in accordance with directions provided on the packaging), to form an opening 130. The sachet 110 may then be rotated on a suitable angle to allow the user to dispense contents of the sachet 110, for example, to add the protein containing substance 100 to the liquid.

In another embodiment, the protein containing substance 100 may be provided in a container including an airtight seal covering an opening of the container. The container may be comprised in or integral with a packaging of the kit 6000, 7000, for example.

In an alternative embodiment, the protein containing substance 100 may be contained within a portion of the sample collection device 200 and may be released into the liquid of the sample during or after collection of the sample. For example, the protein containing substance 100 may be contained within a bulb or stem of a pipette. In other embodiments, the protein containing substance 100 may be contained and/or packaged in any other means suitable for holding and preserving the protein containing substance 100 prior to use, based on the form of the substance as previously disclosed including, for example, a container, capsule, pouch, wrap or bag.

The test device 300 is configured to detect at least one analyte indicative of a pathological condition in the faecal material from which the sample is derived. In one embodiment, the test device 300 is configured to detect at least one analyte representative of occult blood, for example, haemoglobin in the sample. Dispersing and/or dissolving the protein containing substance 100 in the liquid may enhance the sensitivity of the test. For example, without wishing to be bound by theory, it is hypothesised that the protein containing substance 100 may function to stabilise the haemoglobin analyte (the analyte being the protein globin moiety of the haemoglobin molecule) in the sample, for example by preserving its conformation stability. Additionally or alternatively, the protein containing substance may also enhance test sensitivity by functioning to displace the haemoglobin, which tends to bind to the faecal material, from the faecal material into the liquid. This may improve the availability of the haemoglobin in the liquid from which the sample is drawn and therefore may improve detection of haemoglobin and consistency of test results at high levels of dilution. Tests conducted according to the present disclosure have been shown to have a limit of detection (LoD) for detection for blood in water of less than about 0.05 μL/L (or <7.5 ng/ml).

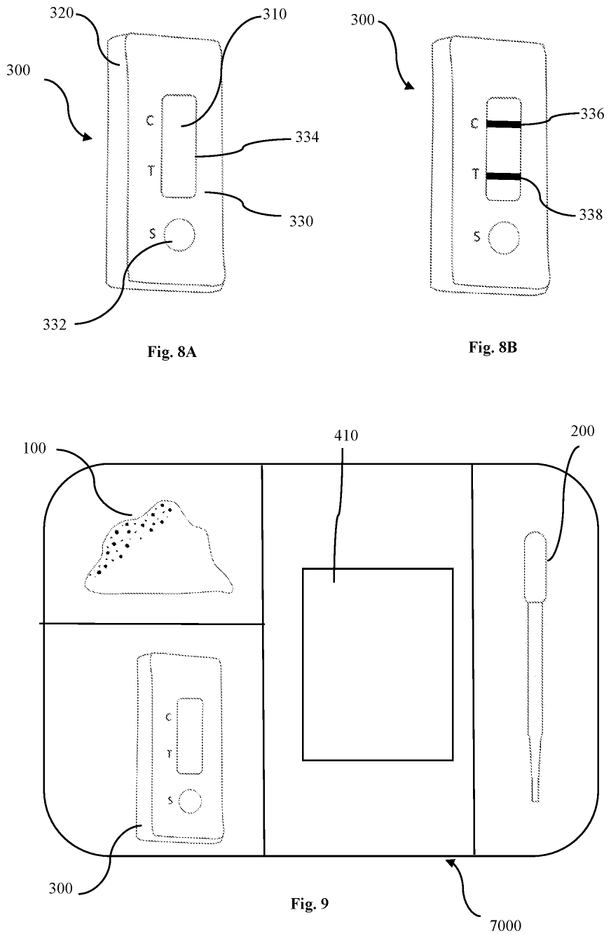

FIGS. 8A and 8B show an embodiment of a test device 300 for inclusion in the kit 6000. In this embodiment, the test device 300 is a faecal immunochemical test device comprising a test strip 310 encased in a housing 320 having a front surface 330. The test strip 310 may be a lateral flow test strip, also known as an immunochromatographic test strip, or immunochemical test strip, known as a “rapid test”. The front surface 330 of the housing 320 may include a first opening defining a sample receiving portion 332. The sample receiving portion 332 may allow access to a portion of the test strip 310 configured for receiving the sample. The front surface 330 of the housing 320 may comprise a second opening, defining a display portion 334. The display portion 334 may be configured to display a results indication portion of the test strip 310. The results indication portion may include a control line 336 and one or more test lines 338. The front surface 330 of the housing 320 may include indicia, indicating the various portions of the test strip. For example, as can be seen in FIGS. 8A and 8B, in this embodiment, the front surface 330 of the housing 320 includes the letters “C”, “T” and “S”, indicating the control line 336, test line 338 and sample receiving portion 332, respectively.

In the illustrated embodiment, the control line 336 and test line 338 include pigmented lines that are visible in the display portion 334, as shown in FIG. 8B. The formation and display of both the control line 336 and test line 338 may indicate a positive test result (i.e. detection of at least one analyte), while the formation and display of only the control line may indicate a negative test result (i.e. no analyte detected). Formation of the test line occurs when conjugated labels and antibodies present in a layer of the test strip 310, such as a nitrocellulose membrane, bind to a target analyte (or analytes) present in the sample. In embodiments where the test device 300 is configured to detect haemoglobin, a positive test result (i.e. the formation and display of both the control and test line as shown in FIG. 8B) indicates the presence of haemoglobin in the sample.

The test device 300 may be deemed faulty in instances where no control line is formed and displayed during testing. The use of a lateral flow test strip 310 allows the test to run and indicate a result in a relatively short period of time following introduction of a sample to the test strip 310, providing an accurate assay of the sample at the time of collection. The test results may be read immediately, or within a specified period of time thereafter, without risk of analyte loss through degradation during transport, as may be found in other tests. The test result may alert the user or attending medic to seek further investigation or testing. In another embodiment, an alternative testing device or assay device suitable for detecting one or more analytes may be used for testing the sample. Additionally or alternatively, testing of the sample may involve transporting the collected sample to a testing facility such as a laboratory for testing.

Another example of a kit 7000 according to the present disclosure is shown in FIG. 9. In this embodiment, the kit 7000 comprises the protein containing substance 100, the sample collection device 200, the test device 300 and the liquid-permeable membrane 410. The liquid permeable membrane 410 may be configured for placement across the liquid to obscure the faecal material after defecation, as described above in relation to test methods 1000A and 1000B.

Additionally or alternatively, the kit 7000 may include the liquid permeable membrane 420 which is configured to sheath the sample collection device 200, as shown and in FIG. 3 and described above.

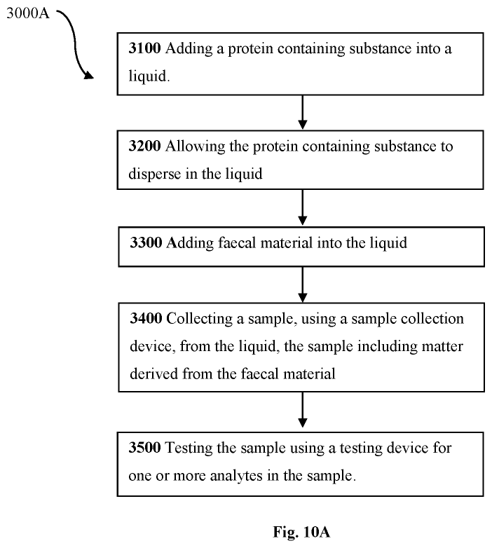

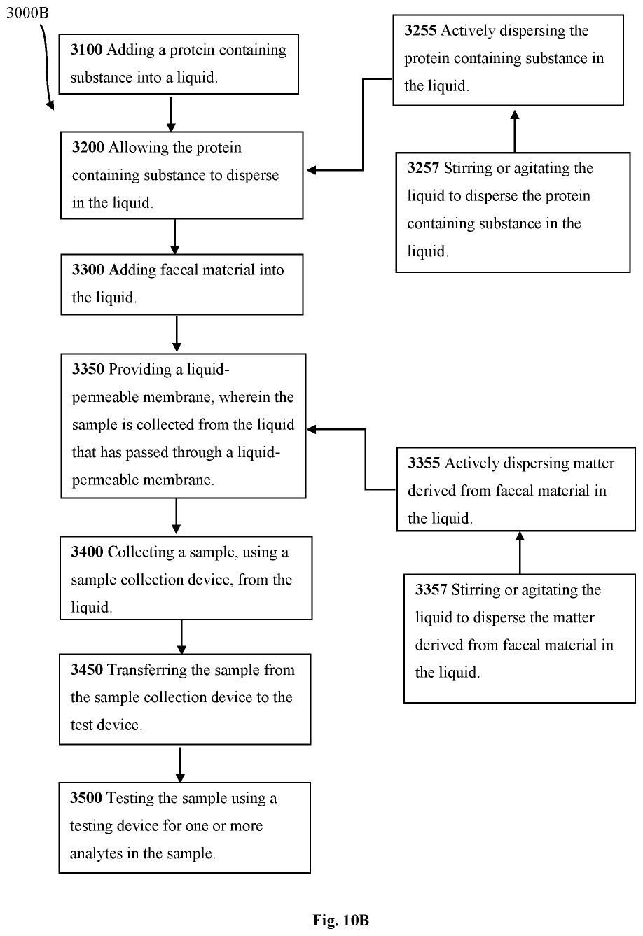

A method for testing a sample derived from faecal material, according to the present disclosure is shown as flowchart 3000A in FIG. 10A. The method 3000A includes adding a protein containing substance into a liquid 3100, allowing the protein containing substance to disperse in the liquid 3200, adding faecal material into the liquid 3300, collecting a sample, using a sample collection device, from the liquid 3400, and testing the sample using a testing device for one or more analytes in the sample 3500.

In some embodiments, the method may comprise one or more further steps 3255, 3257, 3350, 3355, 3357 and 3450, for example as shown in flowchart 3000B of FIG. 10B. The relative positions of the method steps as depicted in flowcharts 3000A and 3000B should not be interpreted as limiting those steps to being performed in any particular order.

The methods may be performed using a test kit, such as kit 6000 or 7000 as described above.

In some embodiments, the liquid may be water, in particular toilet bowl water. In this embodiment, adding the protein containing substance 3100 into the liquid may comprise adding the protein containing substance (e.g. milk powder) into water contained in a toilet bowl. For example, the contents of sachet 100 may be sprinkled over the surface of the toilet water.

Adding faecal material into the liquid 3300 may include adding faecal material into toilet bowl water in a toilet bowl, for example by using the toilet for defecation in the normal fashion. The presence of urine and/or toilet paper in the toilet bowl does not preclude testing and sampling. In another embodiment, however, the liquid may be water in any other container suitable for receiving faecal material. Adding the protein containing substance into the liquid 3100 may comprise adding the protein containing substance into the water in the container. Similarly, in this embodiment, adding faecal material into the liquid 3300 includes adding faecal material into water in the container suitable for receiving faecal material, after defecation. Alternatively, water (or other test liquid) may be added to the container after adding the faecal material and/or protein containing substance 100 to the container. In some embodiments, the protein containing substance 100 is added to the liquid prior to the addition of faecal material in the liquid. This may reduce the risk of user contact with faecal material. However, in other embodiments, the protein containing substance 100 is added to the liquid after the addition of the faecal material to the liquid. In yet another embodiment, the protein containing substance 100 is added to the liquid in the sample after collection of the sample of liquid.

In some embodiments, the method includes actively dispersing the matter derived from faecal material in the liquid 3355 by stirring and/or agitating the liquid (for example, using the sample collection device or another suitable stirring implement) as described above. Stirring or agitating the liquid may enhance dispersion of the matter derived from the faecal material to mix or disperse in the liquid in which the faecal material is submerged. Manipulating the sample collection device 200 may include advancing the sample collection device 200 towards the liquid containing the protein containing substance 100, inserting at least a portion of the distal end of the sample collection device 200 in the liquid and manoeuvring the sample collection device 200, from the proximal end P, in a stirring motion to actively mix and therefore disperse the matter derived from faecal material. In one example, where the sample collection device is a pipette 200 as described above and as shown in FIGS. 2, 3, 6 and 9 the user may hold the pipette at the proximal end P, at the top of the hollow bulb 210 and advance the pipette in the liquid such that the distal end D of the hollow tube 220 is inserted into the liquid for stirring. In another embodiment, any other sample collection device, such as a brush or spoon-like structure, suitable for stirring and agitating the liquid may be used.

The method may further include allowing the protein containing substance 100 to disperse in the liquid. In some embodiments, this may include allowing passive dispersion of the protein containing substance 100 in the liquid over time. In another embodiment, allowing dispersion of the protein containing substance in the liquid includes active dispersion of the protein containing substance 3255 stirring and/or agitating the liquid (for example, using the sample collection device or another suitable stirring implement as previously described) to disperse the protein containing substance 100 in the liquid 3257. While both passive and active dispersion of the protein containing substance 100 may occur, active mixing and stirring of the protein containing substance may further assist in dispersing and/or dissolving the protein containing substance in the liquid. Dispersion may facilitate a more even distribution of the protein containing substance 100 in the liquid. Dispersion may improve interaction of the protein containing substance 100 with the faecal material in the liquid. Increased interaction of the protein containing substance with the faecal material increases the probability that the haemoglobin analyte is stabilised, for example by maintaining conformation stability of the haemoglobin in the collected sample, and may therefore improve detection of haemoglobin at high levels of dilution. Additionally, in an embodiment where the protein containing substance 100 is derived from milk, or includes a pigment or dye capable of increasing the opacity of the liquid, stirring and agitating the liquid containing the protein containing substance may further aid to provide an even distribution of the protein containing substance, therefore increasing the opacity of liquid in a consistent manner which may enhance obscuring of faecal material present in or added to the liquid. In other embodiments, the protein containing substance 100 may not increase the opacity of the liquid following addition and/or dispersion, maintaining a substantially transparent liquid. For example, in some embodiments, the protein containing substance may include BSA.

In one embodiment, dispersing the protein containing substance 100 in the liquid occurs prior to collection of the sample. Dispersing the protein containing substance may occur prior to the addition of faecal material in the liquid. In one example, the protein containing substance 100 may be added to the liquid and allowed to disperse in the liquid prior to the addition of any faecal material in the liquid. To assist in dispersion of the protein containing substance 100 the user may stir and/or agitate the liquid, as described above, prior to the addition of faecal material in the liquid. The faecal material may then be added to the liquid. Stirring and/or agitating the liquid, as described above, may be repeated to disperse the matter derived from faecal material. For example, the stirring and/or agitation may enhance dispersion of occult blood (and therefore haemoglobin) in the liquid. In one embodiment, dispersion of the protein containing substance 100 and the matter derived from faecal material may occur in the same stirring and agitation action.

In another example, the protein containing substance 100 may be added to the liquid and allowed to disperse in the liquid after the addition of faecal material in the liquid. In this instance the dispersion of the protein containing substance 100 and the matter derived from faecal material, i.e. occult blood containing haemoglobin, may occur in the one action. For example, the sample collection device 200 may be manipulated to stir and/or agitate the liquid containing both the protein containing substance 100 and the matter derived from faecal material at the same time. In other embodiments, dispersion of the matter derived from faecal material and the protein containing substance 100 may occur in two separate actions, wherein the matter derived from faecal material is dispersed prior to the protein containing substance 100. In yet another embodiment, dispersing the protein containing substance 100 may occur after collection of the sample and more specifically after the addition of the faecal material in the liquid. In this example, the faecal material may be added to the liquid and the matter derived from the faecal material may be dispersed in the liquid prior to collection of the sample, as previously described. Following collection of the sample using the sample collection device 200, the protein containing substance 100 may then be added to and dispersed in the liquid of the sample within a portion of the sample collection device, for example in the hollow bulb 210 of the pipette 200 as shown in FIG. 2. In this example, the pipette 200 may be manipulated to stir or agitate the liquid of the sample within the hollow bulb 210 by shaking or swirling the pipette.

In one embodiment, the method further includes providing a liquid-permeable membrane 410, 420, wherein the sample is collected from the liquid that has passed through the liquid-permeable membrane as shown in step 3350 of FIG. 6B. In one embodiment, providing the liquid-permeable membrane, for example 410, includes placing the liquid-permeable membrane 410 across the liquid, to at least partially obscure the faecal material. In this embodiment, the liquid-permeable membrane 410 is placed across the liquid, prior to collection of the sample such that it creates a barrier between the liquid and its contents and the user, at least partially obscuring any faecal material contained in the liquid. In some embodiments, the liquid-permeable membrane 410 may be placed across the liquid prior to actively dispersing the matter derived from faecal material in the liquid. In this instance collecting the sample from the liquid involves advancing the sample collection device 200 towards the liquid-permeable membrane 410 and depressing a region of the liquid-permeable membrane 410 into the liquid using a distal end of the sample collection device 200, for example the distal end D or distal tip of pipette 200. The pressure applied to contact and depress a region of the liquid-permeable membrane 410, causes the liquid-permeable membrane to fold around a distal portion, adjacent the distal end D, of the collection device. Folding of the liquid-permeable membrane 410 around the collection device 200 may act as a further barrier between the sample collection device 200 and the faecal material below the liquid-permeable membrane 410 preventing the passage of any solid faecal material. This may optimise the chance that that the user avoids any direct encounter with the faecal material itself.

In another embodiment, providing a liquid-permeable membrane, for example liquid-permeable membrane 410 or 420, may include sheathing at least a distal end D of the sample collection device 200. In this embodiment, the sample collection device is sheathed by the liquid-permeable membrane 410, 420 prior to collection the sample. The liquid-permeable membrane 410, 420 may be configured to partially sheath the sample collection device from the distal end D and/or distal tip of the device or substantially sheath the sample collection device from the distal end D and/or distal tip to the proximal end P of the device. In one embodiment, the sample collection device 200 may be sheathes by the liquid-permeable membrane 420 prior to actively dispersing the matter derived from faecal material in the liquid. In this embodiment, the sample collection device 200, sheathed in the liquid-permeable membrane 420, is advanced toward the liquid for collection of the sample as well as for manipulation of the liquid, prior to collection of the sample.

The liquid-permeable membrane 410, 420 may be configured to fold or wrap around the sample collection device. Additionally or alternatively, the liquid permeable membrane 410, 420 may be in the form of a sleeve or pouch. Advancing the sample collection device 200 sheathed in the liquid-permeable membrane 420 may further include depressing a region of the liquid-permeable membrane 420 when the distal end D and/or distal tip of the sample collection device 200 makes contact with the liquid. The method may include withdrawing the sample collection device 200 from the liquid-permeable membrane 420 prior to testing the sample and in particular prior to transferring the sample to a testing device.

In one embodiment, collecting the sample comprises collecting a volume of the liquid substantially corresponding to a volume required for testing the sample. Testing the sample may comprise transferring the sample from the sample collection device to the test device 3450, for example, by transferring the sample from the pipette 200 to the sample receiving portion 332 of the test device 300. In another embodiment, testing of the sample may involve transporting the sample to a testing facility such as a laboratory for testing.

Testing the sample using the testing device 300 may include testing for one or more analytes indicative of a pathological or other condition in the faecal material from which the sample is derived, for example testing for at least one analyte that is representative of occult blood (such as haemoglobin) in the sample. In one embodiment, the test device 300 comprises a faecal immunochemical test device, embodying principles described above and comprising a lateral flow test strip 310. If haemoglobin is detected in the sample, the test device may display a positive result, alerting the user to the presence of occult blood in the stool, which may be indicative of a pathological condition. This may then prompt the user to seek further medical testing or investigation to further assess and diagnose the source of the bleeding and/or any related pathological condition such as colorectal cancer, pre-cancerous polyps, adenomas or lesions. In another embodiment, an alternative testing device or assay device suitable for detecting the one or more analytes may be used for testing the sample.

Methods according to the present disclosure may provide a relatively simple, inexpensive sampling method with little or no direct stool handling, reducing the possibility of direct contact with the stool and improving acceptability of conducting the test amongst the population. A kit, apparatus or method according to the present disclosure may provide increased sensitivity in comparison to other known FITs and sampling methods increasing the possibility of detection of minute quantities of blood.

Kits and methods according to the present disclosure may increase the possibility of detection of haemoglobin that is representative of the whole stool sample as opposed to a small sample of the stool, as is common with conventional FITs and sampling methods.

The kits, apparatus and methods according to the present disclosure may facilitate monitoring of subjects with very low stool haemoglobin levels that could be at risk of the development of pathologies such as colorectal cancer and precursor polyps. In other examples, test kits, apparatus and/or methods according to the present disclosure may have applications in forensic testing.

It will be appreciated that, while some features may be disclosed in relation to one or more embodiments, and other features be disclosed in relation to one or more other embodiments, combining these features together in one or more further embodiments is within the scope of the disclosure. It would consequently be understood that any combinations of any disclosed features in an embodiment of the methods, apparatuses or kits described herein, is within the scope of the disclosure.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXPERIMENTAL EXAMPLES

### Example 1

In one example, the limit of detection (LoD) of a test kit according to the present disclosure was assessed. In this example, samples were tested for the presence of blood using a CTK FOB-HI rapid test device (CTK Biotech Inc, Poway, CA92064, USA). Samples were collected using a sampling device according to the present disclosure.

30 g of whole milk powder was added to a 3 litre container containing 1 litre of tap water. 10 mL of the liquid, containing the whole milk powder, was collected and reserved. The liquid was then sampled and tested (control negative).

After weighing the container and contents, a naturally passed stool from a healthy subject was added to the container. The container and its contents were then weighed again to determine the weight of the stool. In this example, the stool weight was 124 grams. Using a sample collection device, a sample was collected from the liquid within the container surrounding the stool and tested (control negative).

10 μL of fresh donor blood was collected from a finger prick. The blood was diluted using the 10 mL reserved volume of liquid with milk powder. The diluted blood was then added to the container with the stool to the cumulative volumes of blood indicated in Table 1 below. Samples were collected and tested.

Results were recorded at 10 minutes after sample addition. As shown in Table 1, after 10 minutes of testing the LoD was 0.025 μL blood/litre. After 40 minutes of test development time the results remained the same. The control negative test results remained stable over the 40 minute development time. Maximum detection intensity was reached after the addition of 0.2 μL of blood. The concentration of haemoglobin (Hb) of the donor blood was 148 μg/μL. Therefore, at 0.025 μL of blood the total Hb present in the sample is equal to 148×0.025=3.7 μg. In 1 litre of water the Hb concentration is therefore 3.7 ng/ml. If the Hb was contributed by the stool of 124 g then the amount of Hb/g of stool would equal 3.7/124=0.03 μg/g of stool. This experiment indicated that the kit and testing method according to the present disclosure enabled detection of blood at a concentration of less than 1 μL/litre.

TABLE 1
results of samples collected and tested, recorded
10 minutes after sample addition.

| Blood added (ul) | Result | Comments |
|---|---|---|
| 0 | −ve | Stool remained |
| | | negative |
| 0.01 | −ve | Trace positive later. |
| 0.025 | 2x low +ve | Limit of Detection |
| 0.05 | 2x +ve | |
| 0.1 | 2x +ve | |
| 0.2 | 2x +ve | |

### Example 2A

In another example further tests were conducted to assess contributors that influenced the LoD of the kit and method. In one example, the LoD of blood in liquid and the effect of adding the protein containing substance were assessed.

Blood was added in 2 μL increments up to a cumulative volume of 8 μL to 1 litre of water. A sample was collected after each addition of blood using a sample collection device according to the present disclosure. Each sample was tested using a test device in accordance with the present disclosure.

Blood was not detected in any of the samples, even at the maximum volume of 8 μL of blood. This indicates the instability of Hb in water at higher dilutions.

To determine if the denaturation of Hb was reversible, 1 μL of blood (100 μL of blood diluted at 1/100) was added to 1 litre of water and tested in duplicate. A sample was collected and tested, following the methods previously discussed, yielding a negative result for the presence of Hb. 30 grams of whole milk powder was then added to the water and a sample was collected and tested according to the presently disclosed methods, yielding a negtiave result for the prescence of Hb. These results therefore suggest that the denaturion of the Hb can not be reversed with the addition of a proetin containing substance (i.e. whole milk powder).

### Example 2B

In another example, the LoD of blood added to a liquid containing a protein containing substance was assessed.

Blood, collected from a finger prick, was diluted 1/100 with tap water. 30 grams of whole milk powder was added to 1 litre of water and a sample was taken and tested, using a sample collection device and test device in accordance with the present disclosure. Diluted blood samples were progressively added to the 1 litre of water containing 30 grams of whole milk powder in increments of 0.1 μL up to 0.5 μL, where a sample was taken and tested after each addition, using a sample collection device and test device according to the present disclosure.

The LoD for blood in a 3% milk solution was 0.3 μL blood/litre. At a blood Hb concentration of 148 μg/L, the total amount of Hb at the LoD is equal to 0.3×148=44 μg in 1 litre of water=44 ng/ml. This is approaching the claimed analytical sensitivity for Hb in stabilising buffer for a CTK test device, when tested in lab conditions, which is 25 ng/ml.

### Example 2C

In another example, the LoD of blood added to a stool in liquid was assessed.

A naturally passed stool was added to 1 litre of tap water in a 3 litre container. In this example, the weight of the stool was 148 grams. Blood, collected from a finger prick, was diluted 1/100 with tap water. Diluted blood was added to the 1 litre of water containing the stool sample at 0.1 μL blood increments (10 μL of 1/100) until the LoD was established in duplicate tests. As recorded in Table 2 below, after 10 minutes the LoD was 1 μL of blood/litre of water (1.0 μL), yielding consistent positive results for the presence of Hb. Moreover, after a 4-hour period the final blood addition (i.e. at 1.25 μL) was retested and yielded positive results for the presence of Hb, therefore evidencing that the Hb molecule was stable in the presence of a stool sample for more than 4 hours. The sensitivity of Hb is equal to 1 μL blood at 148 μg/μL=148 μg/litre or 148 ng/ml. For the stool of weight 148 g the Hb/g stool=148/148=1 μg/g. This sensitivity compares favourably with the reported sensitivity of a standard OC-Sensor of 3.8 μg/g of stool.

TABLE 2
Test device results following the cumulative addition
of blood volume in 0.1 μL increments in 1
litre of water containing the stool sample.

| | Cumulative blood | |
|---|---|---|
| Test number | volume (μL) | Result |
| 1 | 0 | Negative |
| 2-7 | 0.1 to 0.5 | Negative |
| 8 | 0.7 | Trace |
| 9 | 0.8 | 1 trace, 1 low + |
| 10 | 0.9 | 2x trace |
| 11 | 1.0 | 3x low positive, 1x trace |
| 12 | 1.25 | Positive |
| 12 after 4 h | 1.25 | Positive |

### Example 2D

In another example, the effect on test LoD with the addition of a protein containing substance to a liquid containing a stool was assessed. In this example, samples were collected using sheathed sample collection devices according to the present disclosure and tested using test devices according to the present disclosure.

A normally passed stool from a healthy subject, with a weight of 232 grams, was added to a 3 litre container containing 1 litre of tap water. A sample was collected using the sample collection device and a test of the sample was conducted using the test device as previously described.

5 μL of 1/100 blood dilution was added to the container comprising 1 litre of water (corresponding to 0.05 μL blood, or about 7.4 ng/ml) and the stool sample. A sample was collected using the sample collection device and a test of the sample was conducted using the test device as previously described. Whole milk powder was then added to the container in 5 gram increments to a maximum of 30 grams. Samples were collected and tested in duplicate after each addition of whole milk powder.

As shown in Table 3 below, a low positive result was returned for the presence of Hb in the sample, following the addition of 5 grams of whole milk powder. Peak intensity reached after the addition of 15 grams of whole milk powder.

These results indicate that the addition of the whole milk powder decreased the LoD and therefore increased sensitivity of the test from 1 μL to 0.05 μL of blood/litre of water, corresponding to a 20-fold increase in sensitivity. Stool added to 30 g milk powder in 1 litre of water decreased the LoD from 0.3 μL to 0.05 μL of blood/litre of water, i.e., a 6-fold increase in sensitivity. This indicates that stool and milk powder may have a complementary effect on the test sensitivity for blood in water.

It was also noted that after a 1-hour period, the test results of test 0, containing no additions of blood or whole milk powder, and test 1, containing a first addition of blood but no milk powder, did not develop any hint of detection of a positive reading and remained negative, hence evidencing the stability of the negative results. A sample, containing the last addition of whole milk powder (i.e. 30 grams total) was taken and retested after a 4 hour period. The Hb was stable for 4 hours in the presence of stool in water containing 3% whole milk powder.

It is apparent that there was a gain in sensitivity from the addition of 5 g (0.5%) of whole milk powder. The addition of 0.05 μL of blood at an Hb concentration of 148 μg/μL provides a Hb concentration of 7.4 ng/ml. The claimed sensitivity of the CTK test device under laboratory conditions is 25 ng/ml. In the present tests the Hb added is equal to 0.05×148=7.4 μg. For a stool of weight 232 g=03 μg/g of stool: =126 times the sensitivity of the OC-Sensor or 116 times the sensitivity for Hb in stool claimed for the CTK test used for in this example of testing the kit and method disclosed herein.

TABLE 3
Test device results following the cumulative addition of
whole milk powder in 5 gram increments in 1 litre of water
containing the stool sample and 0.05 μL of blood.

| Test number | Cumulative whole milk | |
|---|---|---|
| (Duplicates) | powder added (grams) | Result |
| 0 (stool in | 0 | Negative |
| 1 litre H₂O) | | |
| 1 (+0.05 ul blood) | 0. | Negative |
| 2 | 5 | Low positive |
| 3 | 10 | Medium positive |
| 4 | 15 | Strong positive |
| 5 | 20 | Strong positive |
| 6 | 30 | Strong positive |
| 6 Retest after | 30 | Strong positive |
| 4 hours | | (no apparent loss). |

Further testing has indicated that alternative sources of protein, such as whole milk or bovine serum albumin (BSA) may be substituted for the milk powder with similar effects.

The results of the above experimental examples conducted using the kits and methods according to the present disclosure indicate that:
Hb is unstable or undergoes a conformation change at higher dilutions in water, such that it is not detectable by the CTK test device;
A protein-containing substance, such as whole milk powder, stabilises the Hb;
The presence of stool in the liquid stabilises the Hb; and
There is an unexpected synergy arising from the addition of both a protein-containing substance and stool in water, that increases the sensitivity of the test beyond the level with either protein or stool present on their own (in the present examples, to a sensitivity of 0.025 μL/litre). This is well beyond the reported detection limits for Hb in stool for conventional test methods.

When compared to conventional test kits and methods, the kit and method according to the present disclosure under test in the above examples demonstrates greatly improved sensitivity to the presence of Hb.

It will be appreciated that, while some features may be disclosed in relation to one or more embodiments, and other features be disclosed in relation to one or more other embodiments, combining these features together in one or more further embodiments is within the scope of the disclosure. It would consequently be understood that any combinations of any disclosed features in an embodiment of the methods, apparatuses or kits described herein, is within the scope of the disclosure.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A kit comprising:
a protein containing substance for dispersion in a liquid;
a liquid permeable membrane configured for filtering the liquid, the liquid containing faecal material;
a sample collection device, for collecting a sample from liquid that has passed through the permeable membrane, the sample containing matter derived from the faecal material; and
a test device configured to detect one or more analytes in the sample.

2. The kit of claim 1, wherein the protein containing substance is derived from milk.

3. The kit of claim 1, wherein the protein containing substance comprises a powder.

4. The kit of claim 1, wherein the protein containing substance increases the opacity of the liquid.

5. The kit of claim 1, wherein the protein containing substance comprises bovine serum albumin (BSA).

6. A method for testing a sample derived from faecal material, the method including:
adding a protein containing substance to a liquid;
allowing the protein containing substance to disperse in the liquid;
adding faecal material to the liquid;
providing a liquid-permeable membrane configured for filtering the liquid containing the faecal material;
collecting a sample, using a sample collection device, from liquid that has passed through the permeable membrane, the sample including matter derived from the faecal material; and
testing the sample, using a testing device, for one or more analytes in the sample.

7. The method of claim 6, comprising:
placing the liquid-permeable membrane across the liquid containing the faecal material, to obscure the faecal material;
advancing the sample collection device towards the liquid-permeable membrane; and
manipulating the sample collection device to stir or agitate the liquid, such as to disperse matter derived from the faecal material in the liquid,
wherein the liquid-permeable membrane is of sufficient strength to substantially maintain its form during the step of stirring or agitation of the liquid.

8. The method of claim 7, further comprising depressing a region of the liquid-permeable membrane into the liquid using a distal tip of the sample collection device, prior to manipulating the sample collection device to stir or agitate the liquid.

9. The method of claim 6, comprising:
sheathing the sample collection device in the liquid-permeable membrane;
advancing the sample collection device, sheathed in the liquid-permeable membrane, toward the liquid containing the faecal material; and
manipulating the sample collection device to stir or agitate the liquid, such as to disperse matter derived from the faecal material in the liquid,
wherein the liquid-permeable membrane is of sufficient strength to substantially maintain its form during the step of stirring or agitation of the liquid.

10. The method of claim 6, wherein the liquid comprises toilet bowl water.

11. The method of claim 6, further comprising testing the sample to detect the presence of one or more analytes in the sample, wherein at least one of the one or more analytes is indicative of a pathological condition in the faecal material from which the sample is derived.

12. (canceled)

13. The kit of claim 1, wherein the liquid-permeable membrane is configured for placement across the liquid containing the faecal material to at least partially obscure the faecal material.

14. The kit of claim 1, wherein the liquid-permeable membrane is configured for sheathing a distal end of the collection device.

15. The kit of claim 13, wherein the liquid-permeable membrane is configured to obscure the faecal material such that substantially all of the faecal material is hidden from the view of a user collecting the sample.

16. The kit of claim 1, wherein the liquid-permeable membrane is substantially opaque.

17. The kit of claim 1, wherein the liquid-permeable membrane comprises a positively charged material.

18. The kit of claim 1, wherein the liquid-permeable membrane comprises a colour catcher or dye trapping agent.

19. The kit of claim 1, wherein the liquid-permeable membrane is configured to be flushable.

20. The kit of claim 1 wherein at least one of the one or more analytes comprises haemoglobin.

21. (canceled)
